(19) United States
(12) Patent Application Publication
(10) Pub. No.: US 2026/0133201 A1
(43) Pub. Date: May 14, 2026

(54) SYSTEM, METHOD AND COMPUTER PROGRAM PRODUCT FOR ANALYZING FLUORESCENTLY LABELED CIRCULATING EPITHELIAL CELLS

(71) Applicant: Acrocyte Therapeutics Inc., New Taipei City (TW)

(72) Inventors: Ying-Chih CHANG, New Taipei City (TW); Lauren Eleanor LIU, New Taipei City (TW); Lucas WANG, New Taipei City (TW); Jen-Chia WU, New Taipei City (TW)

(21) Appl. No.: 19/387,722

(22) Filed: Nov. 13, 2025

Related U.S. Application Data
(60) Provisional application No. 63/720,368, filed on Nov. 14, 2024.

(51) Int. Cl. G01N 33/58; G01N 21/64; G06T 7/00; G06T 7/13; G06V 10/25; G06V 10/26; G06V 20/69

(52) U.S. Cl. CPC G01N 33/582; G01N 21/6428; G01N 21/6456; G06T 7/0012; G06T 7/13; G01N 2021/6439; G06T 2207/10064; G06T 2207/30024; G06T 2207/30204; G06T 2207/30242; G06V 10/25; G06V 10/267; G06V 20/695; G06V 20/698

(57) ABSTRACT

An analysis system for analyzing an image of fluorescently labeled circulating epithelial cells includes at least a processor. The processor executes a processing procedure, wherein the processing procedure includes steps of: detecting boundaries of multiple cell areas from the image; measuring fluorescence signal intensity of each cell area to calculate expression levels of various cell markers of each cell area; and determining marker statuses of various cell markers of each cell area according to the expression levels of various cell markers of each cell area.

### CROSS-REFERENCE TO RELATED APPLICATION

This application claims the benefit of filing date of U.S. Provisional Application Ser. No. 63/720,368 filed on Nov. 14, 2024 under 35 USC § 119(e)(1), the subject matter of which is incorporated herein by reference.

### BACKGROUND

### FIELD OF THE DISCLOSURE

The present disclosure relates to a system, a method, and a computer program product for analyzing fluorescently labeled circulating epithelial cells.

### Description of Related Art

When the circulating epithelial cells are treated with different antibodies, the cells with expressions of various cell markers may exhibit different reactions to the corresponding antibodies, and these expressions may be visualized using different fluorescent labels. However, there is currently a lack of effective computer-assisted systems for processing and analyzing these fluorescently labeled cells.

Therefore, it is desired to provide an improved analysis system, method, and computer program product so as to improve these issues.

### SUMMARY

An object of the present disclosure is to provide an analysis system for analyzing an image of fluorescently labeled circulating epithelial cells. The analysis system includes one or more processors for executing a processing procedure according to a plurality of instructions, wherein the processing procedure includes the steps of: obtaining an image of plurality of cultured cells which contains circulating epithelial cells; executing an object boundary detection procedure to detect boundaries of multiple cell areas from the image; executing a cell markers expression calculation procedure to measure intensity of multiple fluorescent signals in each cell area to calculate expression levels of multiple cell markers in each cell area; and executing a marker status determination procedure to determine marker statuses of the multiple cell markers in each cell area based on the expression levels of the multiple cell markers in each cell area.

Another object of the present disclosure is to provide an analysis method performed by an analysis system for analyzing an image of fluorescently labeled circulating epithelial cells. The analysis method includes the steps of: culturing a plurality of cells containing circulating endothelial cells (CECs), white blood cells (WBCs), or peripheral blood mononuclear cells (PBMCs); fluorescently labeling and imaging the plurality of cultured cells; executing an object boundary detection procedure to detect boundaries of multiple cell areas from the image; executing a cell markers expression calculation procedure to measure intensity of multiple fluorescent signals in each cell area to calculate expression levels of the multiple cell markers in each cell area; and executing a marker status determination procedure to determine marker statuses of the multiple cell markers in each cell area based on the expression levels of the multiple cell markers in each cell area.

A still another object of the present disclosure is to provide a computer program product stored in one or more non-transitory computer-readable media for enabling an analysis system including one or more processors to analyze an image of fluorescently labeled circulating epithelial cells, wherein the image is obtained by culturing a plurality of cells containing circulating endothelial cells (CECs), white blood cells (WBCs), or peripheral blood mononuclear cells (PBMCs), and fluorescently labeling and imaging the plurality of cultured cells. The computer program product includes: an instruction for causing the one or more processors to execute an object boundary detection procedure to detect boundaries of multiple cell areas from the image; an instruction for causing the one or more processors to execute a cell markers expression calculation procedure to measure the intensity of multiple fluorescent signals in each cell area to calculate expression levels of multiple cell markers in each cell area; and an instruction for causing the one or more processors to execute a marker status determination procedure to determine marker statuses of the multiple cell markers in each cell area based on the expression levels of the multiple cell markers in each cell area.

Other novel features of the disclosure will become more apparent from the following detailed description when taken in conjunction with the accompanying drawings.

### BRIEF DESCRIPTION OF DRAWINGS

FIG. 1 is a system architecture diagram of an analysis system according to an embodiment of the present disclosure;

FIG. 2 is a flowchart illustrating the steps of the sample preparation procedure for circulating epithelial cells according to an embodiment of the present disclosure;

FIG. 3 is a flowchart illustrating the processing procedure of the analysis system according to an embodiment of the present disclosure,

FIG. 4A is a schematic diagram illustrating an image of a cell nucleus presented by a nucleic acid dye and that after executing step S311 according to an embodiment of the present disclosure; and

FIG. 4B is a schematic diagram illustrating an image of a cell marker presented by a single color channel and that after executing step S312 according to an embodiment of the present disclosure.

### DETAILED DESCRIPTION OF EMBODIMENT

Reference will now be made in detail to exemplary embodiments of the present disclosure, examples of which are illustrated in the accompanying drawings. Wherever possible, the same reference numerals are used in the drawings and description to refer to the same or like parts.

Throughout the specification and the appended claims, certain terms may be used to refer to specific components. Those skilled in the art will understand that electronic device manufacturers may refer to the same components by different names. The present disclosure does not intend to distinguish between components that have the same function but have different names. In the following description and claims, words such as “containing” and “comprising” are open-ended words, and should be interpreted as meaning “including but not limited to”.

The terms, such as “about”, “substantially”, or “approximately” are generally interpreted as within 10% of a given value or range, or as within 5%, 3%, 2%, 1% or 0.5% of a given value or range.

In the specification and claims, unless otherwise specified, ordinal numbers, such as “first” and “second”, used herein are intended to distinguish components rather than disclose explicitly or implicitly that names of the components bear the wording of the ordinal numbers. The ordinal numbers do not imply what order a component and another component are in terms of space, time or steps of a manufacturing method. Thus, what is referred to as a “first component” in the specification may be referred to as a “second component” in the claims.

In the present disclosure, the terms “the given range is from the first numerical value to the second numerical value” and “the given range falls within the range from the first numerical value to the second numerical value” mean that the given range includes the first numerical value, the second numerical value, and other numerical values therebetween. In addition, when used in the phrase “between a first value and a second value”, unless otherwise specified, it means “greater than or equal to the first value, and less than or equal to the second value”.

It should be understood that, without departing from the spirit of the present disclosure, in the following embodiments, the features in different embodiments may be replaced, reorganized or mixed to accomplish other embodiments. The features among various embodiments may be mixed and matched arbitrarily as long as they do not violate the spirit of the invention or conflict with each other.

Unless otherwise defined, all terms (including technical and scientific terms) used here have the same meanings as commonly understood by those skilled in the art of the present disclosure. It is understandable that these terms, such as those defined in commonly used dictionaries, should be interpreted as having a meaning consistent with the relevant technology and the background or context of the present disclosure, rather than in an idealized or excessively formal interpretation, unless specifically defined.

In addition, the term “adjacent” used herein may refer to describe mutual proximity and does not necessarily mean mutual contact.

In addition, the description of “when . . . ” or “while . . . ” in the present disclosure means “now, before, or after”, etc., and is not limited to occurrence at the same time. In the present disclosure, the similar description of “disposed on” or the like refers to the corresponding positional relationship between the two components and does not limit whether there is contact between the two components, unless specifically limited. Furthermore, when the present disclosure recites multiple effects, if the word “or” is used between the effects, it means that the effects may exist independently, but it does not exclude that multiple effects may exist at the same time.

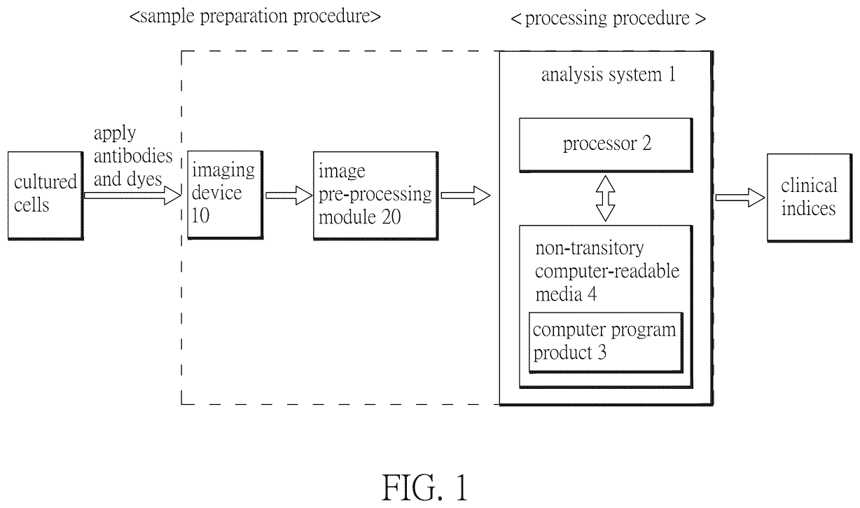

FIG. 1 is a system architecture diagram of an analysis system 1 according to an embodiment of the present disclosure, which illustrates the system architecture of the analysis system 1 in the form of a block diagram. As shown in FIG. 1, the analysis system 1 includes one or more processors 2 and is adapted to synchronously or asynchronously receive a real-time image data. When it is at a multiple-processor state, the one or more processors may be disposed in a distribution manner, for example, one part of the one or more processers are disposed in local device or devices (such as one or more computers or other electronic devices with processors), and the other part of the one or more processers are disposed in a cloud server, while it is not limited thereto. The analysis system 1 may be used in conjunction with an imaging device 10 and an image preprocessing module 20, or in other embodiment, the imaging device 10 may comprise the image preprocessing module 20, or the imaging device 10 and/or the image preprocessing module 20 may be a part of the analysis system 1. Wired communication or wireless communication may be performed between the processor 2 and the imaging device 10. The imaging device 10 is configured to obtain one or more images of fluorescently labeled circulating epithelial cells.

The processor 2 analyzes the one or more images of fluorescently labeled circulating epithelial cells and outputs or generates a clinical index corresponding to each image.

In one embodiment, the processor 2 may execute a processing procedure according to a plurality of instructions of a computer program product 3, thereby processing and analyzing the one or more images of fluorescently labeled circulating epithelial cells. In one embodiment, the computer program product 3 may be, for example, software and may be stored in one or more non-transitory computer-readable media 4. The types of non-transitory computer-readable media 4 may include, but are not limited to, memory, hard drives, flash drives, virtual memory, cloud drives, or other devices with similar functions. In one embodiment, the non-transitory computer-readable media 4 may be disposed in the same device where the processor 2 is disposed, or may also be disposed in different devices.

In one embodiment, the imaging device 10 may be, for example, a camera or a video camera, for capturing images of a plurality of fluorescently labeled circulating epithelial cells, wherein the plurality of fluorescently labeled circulating epithelial cells may be generated through a sample preparation procedure by the image pre-processing module 20. In some embodiments, the analysis system 1 is configured to obtaining an image of a plurality of cells, for example, the processor 2 can obtain the image from the imaging device 10 or the image pre-processing module 20 by wired communication or wireless communication. The image is obtained by culturing a plurality of cells containing circulating endothelial cells (CECs), and optionally white blood cells (WBCs) or peripheral blood mononuclear cells (PBMCs); and fluorescently labeling and imaging the plurality of cultured cells by the analysis system 1 through the imaging device 10 and/or image pre-processing module 20.

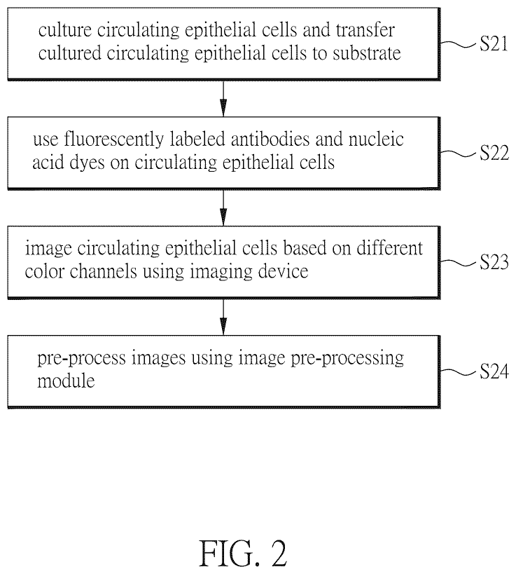

FIG. 2 is a flowchart illustrating the steps of the sample preparation procedure for circulating epithelial cells according to an embodiment of the present disclosure. As shown in FIG. 2 with reference to FIG. 1, step S21 is first performed to culture a plurality of cells of an organism and transfer the plurality of cultured cells containing the circulating epithelial cells to a substrate. Then, step S22 is performed to use a plurality of fluorescently labeled antibodies and one or more nucleic acid dyes on the plurality of circulating epithelial cells. Then, step S23 is performed to image the plurality of cultured cells containing circulating epithelial cells based on different color channels using the imaging device 10 so as to obtain a plurality of images in each of the different color channels. Then, step S24 is performed to pre-process the plurality of images using an image pre-processing module 20.

Regarding step S21, in one embodiment, the organism may be, for example, a healthy subject or patient, and the cultured cells may belong to the same subject or patient. In one embodiment, the plurality of cells may include circulating endothelial cells (CECs), white blood cells (WBCs), or peripheral blood mononuclear cells (PBMCs) obtained from 0.1 to 1.0 ml of blood, but it is not limited thereto. In one embodiment, the substrate may be transparent and include, for example, a glass slide, an elastic film, polystyrene, or other similar materials, but it is not limited thereto.

Regarding step S22, in one embodiment, a nucleic acid dye may be used to dye the cell nucleus, and the type of nucleic acid dye may include fluorescent nucleic acid dyes, such as DAPI and Hoechst, etc., but it is not limited thereto. In one embodiment, the type of antibody used is an antibody that may recognize and attach to a portion of cell markers of epithelial cells, wherein the cell marker corresponding to the antibody may include at least one of EpCAM, PanCK, CD45, PDL1 and HER2, or other suitable cell markers, or any combination thereof, but it is not limited thereto. In one embodiment, the type of fluorescent label of the antibody may include FITC or other suitable fluorescent labels, but it is not limited thereto. In one embodiment, the antibody may be preconjugated or non-preconjugated. The term “antibody” is used broadly and includes genetically engineered forms such as chimeric antibodies and “antigen binding fragment”, which is simply any portion of an antibody that retains the ability to specifically bind its target antigen. In certain labeling methods, the process involves a system where a first antibody binds to a target cell marker (a protein), which is optionally followed by a second, different antibody that is conjugated to a detectable fluorescent label. Crucially, the fluorescent labeling methods used in this context are not limited to identifying only the peptide (protein) portion of the cell marker, but are also designed to target and detect the glyco group (carbohydrate) structures associated with the marker.

Typically, to detect the nucleus in a cell, the nucleus of the cell may be dyed with a nucleic acid dye, which will cause the nucleus to appear in a specific color. Furthermore, to detect a specific cell marker of a cell, an antibody having high affinity to specifically bind to the cell marker may be applied to the cell. The antibody itself is directly conjugated with a fluorescent label (interchangeably denoted as fluorescent marker), so that, when the antibody binds to the cell marker, the antibody with the fluorescent label will mark the areas where the cell markers are located. Next, the areas can be visualized by the fluorescent microscope. Due to the high complexity of biological systems, a single cell marker is often insufficient to reflect the state of a cell. Therefore, it is often necessary to detect multiple cell markers to more accurately determine specific biological phenomena. To effectively distinguish between various protein markers, in one embodiment, different cell markers are distinguished by using a panel of antibodies, each labeled with a fluorophore that features distinct excitation and emission wavelengths (or spectral properties). Therefore, after applying multiple antibodies to the cell, the antibodies will bind to the specific cell markers, and the expressed cell markers will appear in the color of the corresponding fluorophore of the fluorescent label. Furthermore, in one embodiment, in order to effectively capture images exhibiting the fluorescent signals of the cell nucleus and various cell markers, a multi-color channel imaging mechanism is required in the microscope or instrument, corresponding to different emission wavelengths of fluorescent label and nucleic acid dye, to obtain clear and distinguishable images. In other words, the cell nucleus and different cell markers may be displayed in different color channels. For example, the cell nucleus may correspond to a certain color channel, EpCAM and PanCK may correspond to another color channel, and CD45 may correspond to yet another color channel, while it is not limited thereto.

Regarding step S23, the plurality of circulating epithelial cells may be, for example, distributed on the substrate and the area of the substrate may be, for example, between 16 and 50 square millimeters (16 mm²≤area≤50 mm²), while it is not limited thereto. In one embodiment, 25 to 49 images may be taken for the same cluster of cultured cells, each image being approximately between 1 and 4 square millimeters (1 mm²≤image≤4 mm²), the pixel size of each image being approximately between 0.1 and 1.5 square microns (0.1 μm²≤pixel size≤4 μm²), and the bit depth being approximately between 8 and 16 bits, while it is not limited thereto. In one embodiment, the sampling or Z-stacking technique may be used to obtain the optimal focus of the image. In one embodiment, four images of different color channels (for example, white, red, blue and green) may be continuously taken at the same position and stitched and processed in a subsequent stage. In one embodiment, since the field of each imaging is limited, an overall image of the area of interest (ROI) may not be captured in one shot. Therefore, after shooting at one position, the imaging position may be slightly shifted and imaging may be repeated, thereby obtaining multiple slightly offset images. These slightly offset images may be stitched in subsequent pre-processing to form the overall image, thereby allowing the details of the ROI area to be fully captured, but it is not limited thereto.

Regarding step S24, in one embodiment, the image pre-processing module 20 may be implemented by executing specific software on a processor, wherein the image pre-processing module 20 may be set in a local device or a cloud device, but it is not limited thereto. In one embodiment, the image pre-processing module 20 may perform background cleaning on the captured images. The technique for background cleaning may include, for example, at least one of Gaussian smoothing, rolling ball algorithm, image thresholding, and morphological operations (such as dilation or erosion), or other suitable algorithms, or any combination thereof, but it is not limited thereto. In one embodiment, the image pre-processing module 20 may also remove, for example, debris, defects, and contamination from the image using a series of software. In one embodiment, the image pre-processing module 20 may also stitch the images, but it is not limited thereto. After pre-processing, the pre-processed images may be stitched. For example, images corresponding to the same color channel and slightly offset from each other may be stitched into an overall image. Subsequently, the overall images from multiple color channels may be obtained and submitted to the analysis system 1 for subsequent analysis and processing, while it is not limited thereto. Alternatively, the overall image from each color channel may be analyzed and processed separately by the analysis system 1 (that is, each color channel image may be analyzed independently). Furthermore, in this step, the images may be converted to grayscale images (although this conversion may also be performed in subsequent processing). Accordingly, the sample preparation procedure can be understood.

Next, the processor 2 of the analysis system 1 may execute a processing procedure to analyze one or more pre-processed images, wherein each pre-processed image may include information on a plurality of cell areas, but it is not limited thereto. For ease of explanation, the following description will focus on the analysis and processing of a single image.

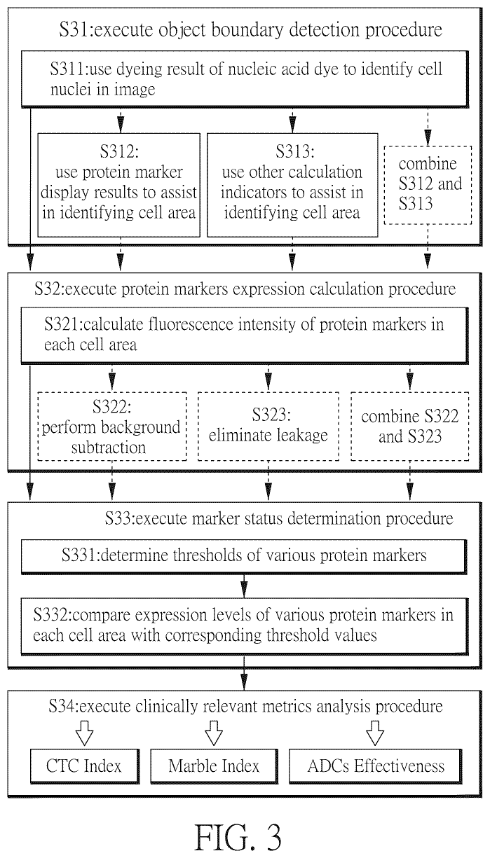

FIG. 3 is a flowchart illustrating the processing procedure of the analysis system 1 according to an embodiment of the present disclosure. As shown in FIG. 3 with references to FIG. 1 and FIG. 2, step S31 is first executed, in which the processor 2 executes an object boundary detection procedure to detect the boundaries of multiple cell areas from an image. Then, step S32 is executed, in which the processor 2 executes a cell markers expression calculation procedure to measure the fluorescence intensity of multiple cell markers in each cell area so as to calculate expression levels of multiple cell markers in each cell area, wherein the cell markers hereby may, for example, be protein markers. Then, step S33 is executed, in which the processor 2 executes a marker status determination procedure to determine marker statuses of the multiple protein markers in each cell area based on the expression levels of the multiple proteins in each cell area. Then, step S34 is executed, in which the processor 2 executes a clinically relevant metrics analysis procedure to output or generate one or more clinical indices corresponding to each cell image based on the expression of the multiple protein markers in each cell image. Therefore, a variety of clinical indices of the subject or patient may be obtained, and these clinical indices may be used to evaluate the clinical condition of the subject or patient, but it is not limited thereto.

Regarding step S31, the goal of the object boundary detection procedure is to find cell areas in the image. That is, after the processor 2 executes step S31, multiple cell areas in the image may be automatically identified.

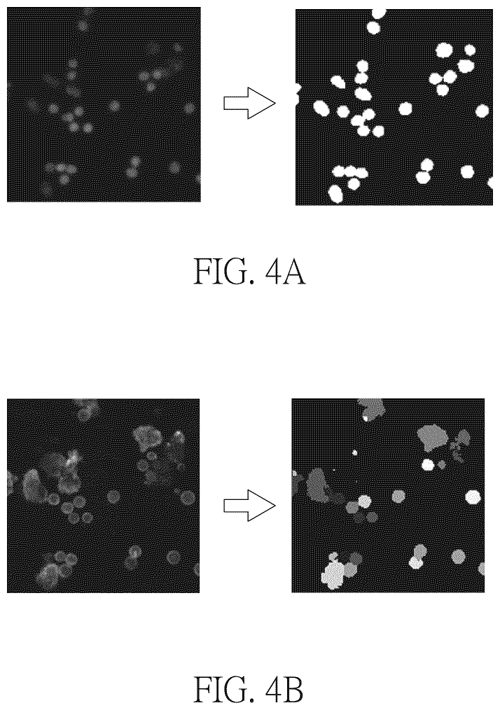

In one embodiment, the object boundary detection procedure of step S31 may include a sub-step S311, wherein sub-step S311 includes: using the dyeing result of the nucleic acid dye as in step S22 to identify one or more cell nuclei in the image. In one embodiment, the identification of the cell nuclei in the image may be achieved by a machine learning model. For example, the processor 2 may use a trained machine learning model to analyze the result of the nucleic acid dye corresponding to the image, and then automatically identify the position of the cell nucleus, while it is not limited thereto. In some embodiments, the type of machine learning model used here may include, for example, a model implemented in software tool such as Stardist or Cellpose, while it is not limited thereto. The machine learning model used here may utilize a portion of the processor 2, but may also be implemented by another processor, or a machine learning model set on a cloud server, while it is not limited thereto. In addition, in another embodiment, the processor 2 may not use a machine learning model, but instead execute a thresholding algorithm (such as, but not limited to, Otsu or minimum cross-entropy) to distinguish the foreground and background in the image. For example, if the foreground is an object with a clear nucleic acid dye, a watershed algorithm is used to distinguish the object, thereby identifying the position of the cell nucleus, but it is not limited thereto. Please refer to FIG. 4A, which is a schematic diagram illustrating an image of a cell nucleus presented by a nucleic acid dye and that after executing step S311 according to an embodiment of the present disclosure. As shown in FIG. 4A, the original image may preliminarily display the dyed area of the cell nucleus and, after executing step S311, the background may be removed, and the cell nucleus may be clearly presented.

Referring to FIG. 3, in one embodiment, the processor 2 may further perform a sub-step S312, wherein step S312 includes: after step S311 is completed, performing background subtraction in conjunction with the protein marker display results (for example, the fluorescent area displayed by the protein marker in response to the antibody applied in step S22) to assist in identifying the cell area in the image. In one embodiment, the processor 2 may perform a threshold algorithm to distinguish the foreground from the background in the image. In this case, the foreground is, for example, an object with one or more protein markers (for example, but not limited to, EpCAM, PanCK, CD45, HER2, and/or PDL1). Then, a watershed algorithm is used to expand the area (for example, to the boundary of the fluorescent area, where the cell nucleus is disposed) with each cell nucleus as the starting point. Then, multiple objects are segmented, and one of the segmented objects may be considered a cell area. In addition, in another embodiment, step S312 may also be implemented using different methods, such as expanding the cell nucleus area according to a preset ratio to form a cell area. For example, an expansion ratio may be preset, and the processor 2 may expand the range of each cell nucleus according to the preset expansion ratio to form multiple cell areas. In one embodiment, the expansion range may also be set to be adjusted according to parameters such as the area of each cell nucleus itself or the Feret diameter, while it is not limited thereto. As shown in FIG. 4B, which is a schematic diagram illustrating an image of a protein marker presented by a single color channel and that after executing step S312 according to an embodiment of the present disclosure. The original image may show the fluorescent area of the protein marker and, after executing step S312, the background may be removed, and the cell area may be clearly presented.

Referring to FIG. 3, in one embodiment, the processor 2 may further execute a sub-step S313, wherein step S313 includes: after step S311 is completed, using one or more calculation indicators to assist in identifying the cell area in the image. In one embodiment, the one or more calculation indicators may include area (number of pixels covered), Feret diameter, eccentricity (the degree to which the shape of the cell area deviates from a circle), proximity to neighboring objects, number of objects within a fixed or variable distance, etc., but it is not limited thereto.

In one embodiment, sub-steps S312 and S313 are selectively executed; that is, whether to execute the sub-steps may be determined according to needs. When sub-steps S312 and S313 are executed together, the results of the two may be mutually verified, or weighted calculations may be performed to generate a final result, while it is not limited thereto. The execution of sub-steps S312 and/or S313 may further improve the identification effect of the processor 2. In addition, in one embodiment, when sub-steps S312 and S313 are not executed, processor 2 may directly use the range of the cell nucleus obtained in step S311 as the cell area and use it in subsequent steps, while it is not limited thereto.

Regarding step S32, the goal of the protein markers expression calculation procedure is to calculate the expression levels of the multiple protein markers in each cell area, such as the expression levels of EpCAM, PanCK, CD45, HER2, and/or PD-L1 in each cell area. That is, after processor 2 executes step S32, the expression levels of the multiple protein markers in each cell area in the image may be calculated. In one embodiment, the image may be converted into a grayscale image, wherein each pixel position of the grayscale image has a grayscale value, but it is not limited thereto.

In one embodiment, step S32 may include a sub-step S321, wherein sub-step S321 includes the following: for each protein marker, the processor 2 may calculate the fluorescence signal intensity in each color channel in each cell area, and define each fluorescence signal intensity as the expression level of the specific protein marker in the cell area. For example, the fluorescence signal intensity in the red channel may correspond to the expression level of one or more protein markers, and the fluorescence signal intensity in the blue channel may correspond to the expression level of another or other protein markers. In addition, these fluorescence signal intensities are converted into grayscale values, but it is not limited thereto. In one embodiment, the processor 2 may define the mean, median, minimum, maximum, 25th percentile, or 75th percentile of the grayscale values of all pixels in the cell nucleus or the entire cell area in each color channel as the expression level of the protein marker, but it is not limited thereto. In one embodiment, the processor 2 may use the mean, median, minimum, maximum, 25th percentile, or 75th percentile of the pixel values (for example, grayscale values) of all pixels in an outer ring of each cell area in various color channels as the protein marker expression level. Here, the outer ring of nucleus or cell area (for example, subtracting out the nucleus area eroded by a fixed value, or by a value dependent on the nucleus'Feret diameter) may be, for example, the area remaining after removing the nucleus from the cell area, but it is not limited thereto. In one embodiment, the processor 2 may also use a mean weighted by a radial function (for example, a Zernike polynomial) to calculate the expression level of a specific protein marker in each cell area, but it is not limited thereto.

In addition to the fluorescence signal intensity (or the grayscale value converted from the fluorescence signal intensity) of the protein marker, images from various color channels may also have some background brightness. This background brightness may arise from protein autofluorescence or camera noise, which may distort the calculated results. Therefore, in one embodiment, step S32 may further include a sub-step S322, wherein sub-step S322 is provided to perform background subtraction to reduce the impact of background pixel values. In one embodiment, for each color channel, processor 2 may obtain a modal pixel value for the entire image (a modal pixel value is, for example, the most common pixel value in the entire image and generally represents the background pixel value), and subtract this modal pixel value from the pixel value corresponding to the protein marker expression obtained in sub-step S321 so as to remove the background. For example, if the pixel value corresponding to the expression of EpCAM in a cell area is 120 and the modal pixel value of the entire image is 10, then after executing sub-step S321, the pixel value corresponding to EpCAM will become 110, and so on. In another embodiment, the processor 2 may find the modal pixel value in each cell area, and then remove the modal pixel value in each cell area.

In addition, in one embodiment, step S32 may further include a sub-step S323, wherein sub-step S323 is provided to eliminate optical leakage between channels. Since different fluorescent dyes may cause optical leakage between different color channels when dyeing cells, sub-step S323 aims to identify and eliminate optical leakage.

In one embodiment, the processor 2 may identify two color channels that may interfere with each other (for example, two channels with adjacent or slightly overlapping wavelength ranges), and then calculate the fluorescence signal intensities of the two color channels in each cell area in the image. Then, linear regression is used to identify the mutual influence of the two color channels and remove the influence.

In another embodiment, interference may be identified by using a control experiment. For example, to remove interference from other color channels from the color channel of the antibody corresponding to CD45, antibodies that are expected to bind to other protein markers (such as EpCAM, PanCK, HER2, and PDL1) may be added first, but antibody that are expected to bind to CD45 (Anti-CD45 antibody) is not added. Then, an image of the cells is captured. In this way, it may be determined that any fluorescent signals appearing in the captured images are caused by the autofluorescence of the cells and the interference. Therefore, the signals caused by the autofluorescence of the cells and the interference may be removed in the images captured later after the antibody corresponding to CD45 is added, while it is not limited thereto.

It should be noted that sub-steps S322 and S323 are optional steps, and may be executed selectively according to needs.

Regarding step S33, the goal of the marker status determination procedure is to determine whether the various protein markers in a cell area are positive or negative based on the expression levels of various protein markers in the cell area. When the interaction between a predetermined antibody and a corresponding protein marker is strong (i.e., shows a high counts or intense fluorescent signal), it means that the expression level of the corresponding protein marker is high, and it also means that the cell area may produce a significant reaction to the predetermined antibody, and vice versa. In one embodiment, step S33 may include sub-steps S331 and S332 that may be executed in sequence, wherein sub-step S331 includes: determining thresholds of various protein markers, and sub-step S332 includes: comparing the expression levels of various protein markers in each cell area with the corresponding thresholds, in which, when the expression level of a predetermined protein marker reaches or exceeds the corresponding threshold, the processor 2 may determine that the protein marker in the cell area is positive, and vice versa, while it is not limited thereto.

Regarding sub-step S331, in one embodiment, a negative control experiment may be performed for various protein markers. For example, when setting up a threshold for a protein marker, by substituting one specific primary antibody with non-binding antibody or buffer, this setup acts as the negative control to accurately determine background signal or noise. A background pixel value for the protein marker is thus determined. Then, the threshold for the protein marker is set based on the background pixel value. In another embodiment, when the area occupied by each cell area in the image does not exceed, for example, 60% of the entire image, a percentile of all pixel values in the image (for example, a PR value) may be set as the threshold. For example, the 85% (PR=85, i.e., a pixel value higher than 85% of the pixel values), the 90% (PR=90, i.e., a pixel value higher than 90% of the pixel values), or the 95% (PR=95, i.e., a pixel value higher than 95% of the pixel values) of all pixel values in the image may be set as the threshold, while it is not limited thereto. In another embodiment, a threshold specific to each cell area may be set for each cell area. For example, a certain percentile of all pixel values in a cell area may be set as the threshold of the cell area, such as 50%, 75%, or 90%, etc., but it is not limited thereto.

It should be noted that the actual threshold corresponding to each protein marker will vary due to variables such as the quality of the antibody supplier's supply, the concentration of the antibody's fluorescent label, the microscope model, the microscope parameter settings, and the characteristics of the cell sample itself. Therefore, the present disclosure only provides a method for setting the threshold, instead of providing the actual value of the threshold.

Regarding sub-step S332, the processor 2 may compare the expression levels of various protein markers in each cell area with corresponding thresholds. Therefore, after executing step S332, the processor 2 may obtain the marker statuses (e.g. positive or negative) of various protein markers in each cell area.

Regarding step S34, the goal of the clinically relevant metrics analysis procedure is to use the marker statuses of various protein markers in each cell area in the image to output/generate one or more clinical indices corresponding to the image by processor 2, so as to obtain one or more clinical indices. In one embodiment, the one or more clinical indices may include, but are not limited to, circulating tumor cells (CTC) index, marble index, and antibody-drug conjugates (ADC) effectiveness. In one embodiment, different clinical indices may be derived by different analysis methods.

Regarding the CTC index, the CTC index may represent the number of CTC cells, and the number of CTC cells is related to the severity of the disease. For example, a greater number of CTC cells generally indicates a more severe disease or a worse prognosis. Therefore, clinical indices related to the CTC index may be used to assist in determining the condition or prognosis of a subject or patient.

In one embodiment, the necessary attributes for the CTC index include identified cell nuclei, and types of CD45, EpCAM and PanCK. The cell nuclei are identified by the nucleic acid dyeing (for example, DAPI+/Hoechst+) as in S31. In addition, marker status, alternatively denoted as phenotype, of the protein markers related to the CTC index include CD45− and EpCAM+/PanCK+, wherein the symbols “+” and “−” indicate marker status of protein markers being positive and negative respectively. For example, CD45-means that CD45 is negative, and so on. In one embodiment, the processor 2 may locate cell areas in the image that have the characteristics of DAPI+/Hoechst+, CD45−, and EpCAM+/PanCK+at the same time, mapping cell areas with the necessary attributes, and calculate the number of the cell areas having the necessary attributes as the CTC index. In one embodiment, the size of an image is approximately 7 square millimeters (mm²), and the CTC index may generally be between 0 and 1500 cell areas (0≤CTC index≤1500), while it is not limited thereto. In this way, the analysis system 1 may execute detection, calculation and analysis procedures to obtain the CTC index corresponding to the image of the subject or patient, and further estimate the condition and prognosis of the subject or patient, but it is not limited thereto.

Regarding the marble index, studies have shown that certain non-adherent cells (for example, epithelial tumor cells) selectively proliferate and form multicellular spheroids. In contrast, suspension cells (for example, white blood cells) are less likely to form multicellular spheroids. Therefore, the phenomenon of multicellular spheroids is a key characteristic of certain tumor cells and highly representative as in vitro model for studying cancer and oncology, and the number of cells in a multicellular spheroid may be considered a marble index. A higher marble index indicates a greater number of cancer cells. Therefore, the marble index may be used as an advanced cancer detection indicator to effectively distinguish cancer samples from healthy samples. Therefore, the clinical indices of the marble index obtained in step S34 at least may be used to assist in cancer detection.

In one embodiment, the necessary attributes for the marble index include identified cell nuclei and types of EpCAM and/or PanCK. The cell nuclei are identified by the nucleic acid dyeing (for example, DAPI+/Hoechst+) as in S31. In addition, types of protein markers related to the marble index may include EpCAM and/or PanCK and marker status thereof is positive (EpCAM+/PanCK+), whereby “the cell areas with EpCAM+/PanCK+characteristics” represents that epithelial cells may be tumor cells and define a cytoplasm area. In one embodiment, the processor 2 may preliminarily screen out cell areas in the image that have both DAPI+/Hoechst+and EpCAM+/PanCK+ characteristics and use them as pre-selected marble cell areas as in S31 and S32. Since whether the pre-selected marble cell areas are multicellular spherical aggregates still needs to be judged based on the actual appearance characteristics of the cells, such as morphology, size, density and/or boundaries, the processor 2 will also use multiple trained machine learning models to assist in determining whether the pre-selected marble cell areas are actual marble cell areas. The machine learning model here may be, for example, a portion of the processor 2, or may be implemented by other processors, or may be set on a cloud server, while it is not limited thereto. Clinically, an increased N: C ratio is a common and important feature defined by the nuclei area and the cytoplasm area and further used to identify abnormal conditions, particularly precancerous dysplasia and various forms of malignant cells.

In one embodiment, the machine learning model may be a supervised learning model. In one embodiment, one or more trained gradient boosting classifiers or random forest classifiers may be used to perform a preliminary analysis based on the morphological characteristics of the pre-selected marble cell area, and output a Boolean value (which may be regarded as a preliminary analysis result) to determine whether it is a marble cell area. Then, one or more trained convolutional neural networks or support vector machines are used to convert the marble cell area of the preliminary analysis result into a numerical output value between 0 and 1. In some embodiments, the output value greater than or equal to 0.5 indicates an actual marble cell area, and vice versa. The above numerical output values are only examples and may be adjusted according to actual needs.

In another embodiment, the machine learning model may be an unsupervised learning type model, such as a machine learning model that uses K-means clustering or principal component analysis (PCA), but it is not limited thereto.

In one embodiment, the data input into the machine learning model for analysis may include, in addition to the pre-selected marble cell area (image) itself, information on characteristics such as the size, roundness, and edge clarity of each cell area, or information such as the expression level of any combination of protein markers, while it is not limited thereto.

Accordingly, the analysis system 1 may obtain the marble index by analyzing the images of the subject or patient, and results of detection of the cancer condition, but it is not limited thereto.

Regarding the ADCs effectiveness as in FIG. 3, antibody-drug conjugates (ADCs) are a new type of cancer treatment with great potential. For example, ADCs may bind to protein markers related to cancer cells (such as HER2 and PD-L1, etc.), so as to directly deliver drugs to target cancer cells. However, when the target antigen is lacking on the surface of cancer cells or the target antigen is expressed at a low level, ADCs may not effectively bind to the cells, which may also result in poor treatment effects. Therefore, the clinical indices related to the effectiveness of ADCs obtained in step S34 at least may be used to assist in evaluating the effects after applying the ADC, such as pre-evaluating whether an ADC may bind to cells.

In one embodiment, a cell area with HER2+ and PD-L1+ characteristics may be first searched from a CTC candidate cell area, wherein the CTC candidate cell area may be determined by using the marker statuses of various protein markers in each cell area in the image to generate the CTC index as described previously. For example, a cell area with the necessary attributes for the CTC index may be selected as the CTC candidate cell area.

Next, using one or more computational metrics (for example, area (number of pixels covered), Feret diameter, eccentricity (degree of shape deviation), proximity to neighboring objects, number of objects within a fixed or variable distance, boundary detection, etc.) and the expression of protein markers (for example, HER2 and PD-L1), ADC effectiveness grade corresponding to the cell area with HER2+ and PD-L1+ characteristics within the CTC candidate cell area is analyzed. The analysis process for HER2 will be described below, and the analysis process for other markers may be deduced similarly.

First, the cell areas with HER2+ characteristics (denoted as HER2+ cell areas) were clustered according to the HER2 expression level in the cell areas. The clustering method here is described as follows:
In a method 1, multiple cutoffs are selected for expression level of HER2 to divide the HER2+ cell areas into multiple bins. For example, based on the expression level of HER2 in each HER2+ cell area, several cutoffs, such as low, medium, and high expression, may be set to cluster the cell areas. In one embodiment, the spacing between the cutoffs is equidistant, but may also be non-equidistant. In one embodiment, the multiple cutoffs for HER2 expression may be linear or non-linear.
In a method 2, multiple critical values of HER2 expression in combination with the expression of other protein markers (such as EpCAM) are selected to divide the HER2+ cell areas into multiple grade intervals. For example, based on the expression of HER2 and EpCAM in each HER2+ cell area, several dividing points, such as low, medium, and high expression, may be set to cluster the cell areas. In one embodiment, the spacing between the dividing points is equidistant, but it may also be non-equidistant. In one embodiment, the multiple critical values of HER2 expression and the multiple critical values of EpCAM expression may be linear or non-linear.

Next, a trained machine learning model is used to analyze the clustered cell areas to evaluate the ADCs effectiveness corresponding to the images. In one embodiment, the machine learning model may be a portion of the processor 2, implemented by another processor, or disposed on a cloud server, but it is not limited thereto. In one embodiment, the machine learning model may be a supervised learning or unsupervised learning model.

In one embodiment, the data input into the machine learning model may include, but are not limited to, the number of cells in each cluster of the HER2+ cell areas, statistical summaries of each cell cluster (for example, the mean, median, 25th percentile (PR=25), or 75th percentile (PR=75) of the expression of protein markers such as HER2 and EpCAM), the correlation or level of unexplained correlation between HER2 and other protein markers, and/or morphological characteristics of the cell areas (for example, area, degree to which the cell deviates from a circle, etc.). The machine learning model may analyze the data to evaluate of ADCs effectiveness.

As a result, the analysis system 1 may determine ADCs effectiveness and further assist in determining the therapeutic effect of ADCs on the subjects or patients, but it is not limited thereto.

Accordingly, the processing procedure of the analysis system 1 is consequently realized and effects obtainable through specific examples of the present disclosure can be understood not being limited to the effects listed above. For example, there may be various technical effects that a person having ordinary skill in the related art can understand or derive from the present disclosure. Accordingly, the specific effects of the present disclosure are not limited to those explicitly described in the present disclosure, and may include various effects that can be understood or derived from the technical features of the present disclosure.

In one embodiment, the present disclosure may determine whether a product in contention falls within the protection scope of the present disclosure at least by the presence or absence of components, component configurations, mechanism observation and/or operating modes of the product to determine whether it falls within the protection scope of the present disclosure, while it is not limited thereto. In addition, if the execution steps of the system involve software, the determination may be made at least by reverse engineering, while it is not limited thereto.

The features among various embodiments may be mixed and matched arbitrarily as long as they do not violate the spirit of the invention or conflict with each other.

Thus, the present disclosure may provide an effective computer-assisted technology to process and analyze fluorescently labeled circulating epithelial cells.

The aforementioned specific embodiments should be construed as merely illustrative, and not limiting the rest of the present disclosure in any way.

1. An analysis system for analyzing an image of fluorescently labeled circulating epithelial cells, comprising:
one or more processors for executing a processing procedure according to a plurality of instructions, wherein the processing procedure includes the steps of:
obtaining an image of a plurality of cultured cells;
executing an object boundary detection procedure to detect boundaries of multiple cell areas from the image;
executing a cell markers expression calculation procedure to measure intensity of multiple fluorescent signals in each cell area to calculate expression levels of multiple cell markers in each cell area; and
executing a marker status determination procedure to determine marker statuses of the multiple cell markers in each cell area based on the expression levels of the multiple cell markers in each cell area.

2. The analysis system as claimed in claim 1, wherein the step of obtaining an image of a plurality of cells includes culturing a plurality of cells containing circulating endothelial cells (CECs), and optionally white blood cells (WBCs) or peripheral blood mononuclear cells (PBMCs); and fluorescently labeling and imaging the plurality of cultured cells to obtain the image.

2. The analysis system as claimed in claim 1, wherein the processing procedure further includes the step of executing a clinically relevant metrics analysis procedure to generate or output one or more clinical indices corresponding to the image based on the marker statuses of the multiple cell markers in each cell area.

3. The analysis system as claimed in claim 2, wherein the one or more clinical indices include at least one of CTC index, marble index, and ADCs effectiveness.

4. The analysis system as claimed in claim 2, wherein the multiple cell markers include at least one of EpCAM, PanCK, CD45, HER2, and PD-L1.

5. The analysis system as claimed in claim 2, wherein the object boundary detection procedure further includes the sub-step of: using dyeing results of one or more nucleic acid fluorescent dyes to identify one or more cell nuclei in the image.

6. The analysis system as claimed in claim 5, wherein the object boundary detection procedure further includes the sub-step of: using each cell nucleus as a starting point to expand and form multiple cell areas.

7. The analysis system as claimed in claim 5, wherein the object boundary detection procedure further includes the sub-step of: using one or more calculation indicators to assist in identifying a cell area corresponding to each cell nucleus, wherein the one or more calculation indicators include at least one of area, Feret diameter, eccentricity, proximity to neighboring objects, and number of objects within a fixed or variable distance.

8. The analysis system as claimed in claim 2, wherein the cell markers expression calculation procedure further includes the sub-steps of: obtaining a modal pixel value of the image; and subtracting the modal pixel value from all pixel values in the image.

9. The analysis system as claimed in claim 2, wherein the marker status determination procedure further includes the sub-steps of: setting corresponding thresholds for various cell markers; and comparing expression levels of the various cell markers in the cell area with the corresponding thresholds, and determining the marker statuses of the various cell markers in the cell area based on comparison results.

10. An analysis method performed by an analysis system for analyzing an image of fluorescently labeled circulating epithelial cells, comprising the steps of:
culturing a plurality of cells containing circulating endothelial cells (CECs), white blood cells (WBCs), or peripheral blood mononuclear cells (PBMCs);
fluorescently labeling and imaging the plurality of cultured cells;
executing an object boundary detection procedure to detect boundaries of multiple cell areas from the image;
executing a cell markers expression calculation procedure to measure intensity of multiple fluorescent signals in each cell area to calculate expression levels of the multiple cell markers in each cell area; and
executing a marker status determination procedure to determine marker statuses of the multiple cell markers in each cell area based on the expression levels of the multiple cell markers in each cell area.

11. The analysis method as claimed in claim 10, further comprising the step of executing a clinically relevant metrics analysis procedure to generate one or more clinical indices corresponding to the image based on the marker statuses of the multiple cell markers in each cell area to obtain one or more clinical index.

12. The analysis method as claimed in claim 11, wherein the one or more clinical indices include at least one of CTC index, marble index, and ADCs effectiveness.

13. The analysis method as claimed in claim 11, wherein the multiple cell markers include at least one of EpCAM, PanCK, CD45, HER2, and PD-L1.

14. The analysis method as claimed in claim 11, further comprising the sub-step of using dyeing results of one or more nucleic acid fluorescent dyes to identify one or more cell nuclei in the image.

15. The analysis method as claimed in claim 14, further comprising the sub-step of using each cell nucleus as a starting point to expand and form multiple cell areas.

16. The analysis method as claimed in claim 14, further comprising the sub-step of: using one or more calculation indicators to assist in identifying a cell area corresponding to each cell nucleus, wherein the one or more calculation indicators include at least one of area, Feret diameter, eccentricity, proximity to neighboring objects, and number of objects within a fixed or variable distance.

17. The analysis method as claimed in claim 11, further comprising the sub-steps of: obtaining a modal pixel value of the image; and subtracting the modal pixel value from all pixel values in the image.

18. The analysis method as claimed in claim 11, further comprising the sub-step of: setting corresponding thresholds for various cell markers; and comparing expression levels of the various cell markers in the cell area with the corresponding thresholds, and determining the marker statuses of the various cell markers in the cell area based on comparison results.

19. A computer program product stored in one or more non-transitory computer-readable media for enabling an analysis system including one or more processors to analyze an image of fluorescently labeled circulating epithelial cells, wherein the image is obtained by culturing a plurality of cells containing circulating endothelial cells (CECs), white blood cells (WBCs), or peripheral blood mononuclear cells (PBMCs), and fluorescently labeling and imaging the plurality of cultured cells, wherein the computer program product comprising:
an instruction for causing the one or more processors to execute an object boundary detection procedure to detect boundaries of multiple cell areas from the image;
an instruction for causing the one or more processors to execute a cell markers expression calculation procedure to measure the intensity of multiple fluorescent signals in each cell area to calculate expression levels of multiple cell markers in each cell area; and
an instruction for causing the one or more processors to execute a marker status determination procedure to determine marker statuses of the multiple cell markers in each cell area based on the expression levels of the multiple cell markers in each cell area.

20. The computer program product as claimed in claim 19, further comprising: an instruction for causing the one or more processors to execute a clinically relevant metrics analysis procedure to generate one or more clinical indices corresponding to the image based on the marker statuses of the multiple cell markers in each cell area to obtain one or more clinically indices, wherein the one or more clinical indices include at least one of CTC index, marble index, and ADCs effectiveness.
